(19) United States
(12) Patent Application Publication
(10) Pub. No.: US 2018/0177644 A1
(43) Pub. Date: Jun. 28, 2018

(54) Diaper Attachment Pod

(71) Applicant: Raja Singh Tuli, Montreal (CA)

(72) Inventor: Raja Singh Tuli, Montreal (CA)

(21) Appl. No.: 15/388,278

(22) Filed: Dec. 22, 2016

(51) Int. Cl. A61F 13/42; A61F 13/15

(52) U.S. Cl. CPC A61F 13/42; A61F 2013/424; A61F 13/15756

(57) ABSTRACT

A system for sensing wetness in an ordinary diaper comprises a disposable sensor strip that adhered to a diaper's padding, and a portable yet non-disposable alerting device. The sensor strip has electrical circuit with conductive elements as wetness sensors, wherein a closed electrical circuit is formed when an amount of body fluid is present that subsequently triggers the alerting device. The sensor strip is electrically coupled to the alerting device with a portion wrapped around the alerting device, wherein the position of the wrapped portion is secured by the indentation or ribs around the outer surface of the alerting device. Locations of the adhesives on the sensor strip are designed in a way that the alerting device is fastened to the outer surface of the diaper without skin contact to the wearer and without contact to any adhesive, such that the alerting device is ready for immediate reuse.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM (EFS-WEB)

Not Applicable

### STATEMENT REGARDING PRIOR DISCLOSURES BY THE INVENTOR OR A JOINT INVENTOR

Not Applicable

### BACKGROUND

### Field of the Invention

This invention relates to devices for monitoring wetness, particularly in diapers, wherein an alerting device is fastened to the outer surface of any ordinary diaper with zero contact to adhesive material that is ready for immediate reuse.

### Description of Related Art

For many years, a variety of designs has been developed for detecting and signalling the presence of urine in a diaper. However, most of these designs required modifications on ordinary diapers in order to adept to such system, such as a pouch like insert is needed in the diaper to house an alerting device as disclosed in U.S. Pat. No. 8,884,769, or a multiple button seats are needed on the outside surface of a diaper to fasten the alerting device as discloses in U.S. application Ser. No. 13/064,832, or embedding the urine sensing electrodes in between different layers of the diaper padding as discloses in U.S. Pat. No. 9,291,589.

In some other cases, the alerting device that gives out signal of a soaked diaper has skin contact with the wearer that may arouse discomfort to the wearer and hygiene concern, for example, U.S. Pat. No. 4,106,001 discloses an alerting device that is clapped over the waistband of the diaper that is in direct skin contact with the wearer.

Further, the wetness sensor and the alerting device are configured as non-detachable in some prior art systems, wherein the entire device must be discarded when the wetness sensor becomes worn. Thus, such alerting devices become relatively expensive to manufacture.

U.S. Pat. No. 5,838,240 discloses a wetness sensing and alerting system that are detachable from each other with the alerting device being adhesively bond to the outside surface of a diaper. While this system is useful as a wetness sensing and alerting system, it is dissimilar to the invention described herein, and lacks certain features and functional benefits, such as a need to clean up any left behind adhesive residue prior next use, which would be apparent to those skilled in the art by reading the detailed description accompanying with the drawings and the claims below.

### BRIEF SUMMARY OF THE INVENTION

The invention described herein depicted a wetness sensing and alerting system comprising an alerting device and a sensor strip. It is intended that the present invention is used on ordinary diaper that needs no further modification. One object of the present invention is to provide a method to attach the alerting device to any ordinary diaper, wherein the alerting device has no contact with any adhesive materials such that it is ready for immediate use when detached from a soaked diaper.

Another object of the present invention is to provide a urine sensing and alerting system that causes minimal discomfort to the user while the portable alerting device has no direct skin contact with the wearer, which also help to maintain the alerting device hygienically clean for reuse purposes.

According to an aspect of the present invention, the sensor strip has electrical circuit printed on its front side while its back side has double-sided adhesives in discrete portions that run across the width of the sensor strip, which allow affixing the sensor strip to a diaper padding such that the present invention covers a detection area from the groin to the hip of a wearer. A temporary yet resilient electrical connection is established between the sensor strip and the alerting device such that the wetness data sensed from the sensor strip is processed by the alerting device to provide an alarm signal or the like by wireless means. By monitoring the current flow or change of voltage in the electrical circuit printed on the sensor strip, the degree and/or locations of wetness of the diaper that caused by urine or other body fluid is detected. The alerting device has indentations or ribs on its outer surface that prevent sideways movement of the sensor strip, as well as a snap lid with locking means to secure the position of the sensor strip in place, which subsequently, to secure the electrical connection between the alerting device and the sensor strip. A sufficient long portion of the sensor strip is wrapped over the alerting device for at least a turn and half in a circular motion, rotating clockwise towards the front waistband of the diaper. The alerting device is fastened to the outer surface, near the center of the diaper's front waistband by the double-sided adhesives located only on the back side of the sensor strip. The sensor strip is designed to be disposable with the soaked diaper while the alerting device is ready for immediate reuse by unwrapping the sensor strip since the alerting device has no contact with the adhesives on the sensor strip.

In another embodiment, multiples double-sided adhesives are placed closed to each other with individual protective film to allow better position control in fastening the alerting device to the outer surface of the diaper waistband.

In another embodiment, the double-sided adhesives are located on both sides of the sensor strip at specific locations when the alerting device is wrapped with the sensor strip in a circular motion that rotates in an anti-clockwise direction.

In another embodiment, the double-sided adhesives are aligned along the length of the sensor strip at certain locations, wherein the locations are chosen that do not impede the urine flow in the diaper padding or reduce the conductivity of the electrical circuit in the sensor strip.

In another embodiment, the double-sided adhesives are presented as continuous strips that run across the width or along the length of the sensor strip.

### BRIEF DESCRIPTION OF THE DRAWINGS

The various preferred embodiments of the present invention described herein can be better understood by those skilled in the art when the following detailed description is read with reference to the accompanying drawings. The components in the figures are not necessarily drawn to scale and any reference numeral identifying an element in one drawing will represent the same element throughout the drawings.

The figures of the drawing are briefly described as follows:

FIG. 1A illustrates a plan view of a back side of a sensor strip of the present invention in a principal embodiment.

FIG. 1B illustrates a plan view of a front side of a sensor strip of the present invention in a principal embodiment.

FIG. 2A illustrates a perspective view of an alerting device in the principal embodiment when its lid is opened.

FIG. 2B illustrates a plan view of an alerting device in the principal embodiment when its lid is closed.

FIG. 3 illustrates a perspective view of a sensor strip affixes to a diaper in the principal embodiment.

FIG. 4 illustrates a perspective view of connecting an alerting device with a sensor strip in the principal embodiment.

FIG. 5A illustrates a plan view of wrapping a sensor strip around an alerting device in a clockwise direction towards the waistband of a diaper.

FIG. 5B illustrates a front view of an alerting device fastened to the diaper's front waistband.

FIG. 6A illustrates a section view of the locations of the double-sided adhesives when an alerting device is wrapped by a sensor strip in a circular motion, rotating in a clockwise direction towards the waistband of a diaper.

FIG. 6B illustrates a plan view of a back side of a sensor strip with adhesives on individual protective film.

FIG. 7A illustrates a plan view of wrapping a sensor strip around an alerting device in an anticlockwise direction towards the waistband of a diaper.

FIG. 7B illustrates a section view of the locations of the double-sided adhesives when an alerting device is wrapped by a sensor strip in a circular motion, rotating in an anticlockwise direction towards the waistband of a diaper.

FIG. 8 illustrates a plan view of a front side of a sensor strip with adhesives at the back that positioned along the length of the sensor strip.

### DETAILED DESCRIPTION OF THE INVENTION

The invention described herein is designed to work along with an ordinary diaper, for people having medical problems such as bladder incontinence or for babies. The present invention discloses a urine detecting and signalling system comprising a disposable wetness sensor strip and a portable alerting device, preferably a non-disposable device. The purpose of the present invention is to provide a method to attach the reusable alerting device to any ordinary diaper without any alteration, wherein the alerting device has no contact with any adhesive materials at all time such that it is ready for immediate use when detached from the soaked diaper.

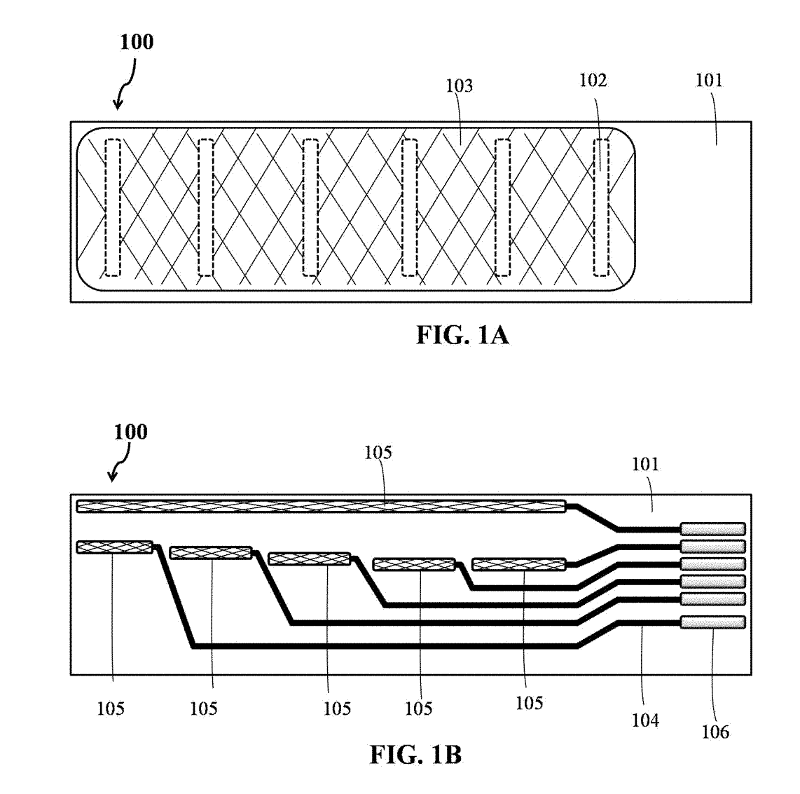

FIG. 1A illustrates a plan view of a disposable wetness sensor strip 100 in a principle embodiment, which basically is a flexible elongate piece of water absorbing material 101, preferably made of low cost materials such as cloth or paper so as to be used only once and be disposed with the soaked diaper. The back side of the sensor strip 100 is a peel-and-stick backing with non-conductive double-sided adhesive 102 materials at certain locations, which are covered by a protective film 103 prior to use. When in use, the protective film 103 is peeled off such that the sensor strip 100 can be adhesively bonded to a diaper. On the front side of the sensor strip 100 as illustrated in FIG. 1B, there is an electrical circuit comprises multiple elongated, spaced apart and generally parallel conductive bands 104 printed in conductive carbon ink. At the rear end of each of the conductive bands 104 are electrodes 105 that act as the wetness detectors, which may also be printed in conductive carbon ink. The multiple electrodes 105 are distributed along the longitudinal direction of the sensor strip 100. The front end of each of the conductive bands 104 is terminated with a contact terminal 106, which may make of silver foil or other conductive materials, to facilitate making an electrical connection with an external alerting device so that wetness data sensed by the electrodes 105 in the sensor strip 100 can be further analysed.

The pattern of the conductive bands 104 and the locations of the electrodes 105 as shown in FIG. 1B is merely one non-limiting example while other alternatives are also conceivable to a person skilled in the art. Further, the length and the width of the conductive bands 104 and the electrodes 105 are adjusted according to the variation in diaper size.

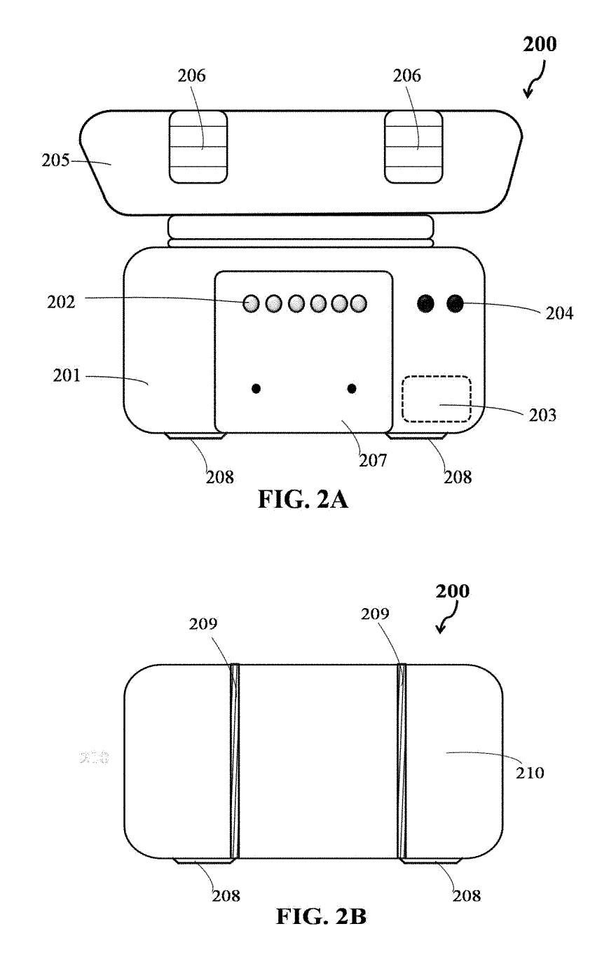

Referring to FIG. 2A now, wherein the portable alerting device 200 has an insulating housing 201, preferably made of plastic, comprises printed circuit board (not shown) with equal number of contact terminals 202 as the sensor strip 100, battery 203 and other electronic components 204. The alerting device 200 is adapted to receive and process signals pick up from the sensor strip 100 and, subsequently, it provides an alarm signal or the like by wireless means. For example, the alarm signal may include sending a text message to a caretaker's cell phone with information about the degree of wetness and the location(s) of wetness on the diaper. The battery 203 provides electric power to the electronic components 204, as well as supplies power to the electrical circuit on the sensor strip 100. In the principle embodiment, the alerting device 200 has a snap lid 205 to protect the contact terminals 202 and to secure the sensor strip 100 once it is inserted inside the alerting device 200. The snap lid 205 is securely closed by further locking the tabs 206 into the locking grooves 208. The width and the thickness of the sensor strip 100 is designed to be fit into an indented area 207 of the alerting device 200 so that the sensor strip 100 does not affect the snap lid 205 to close. Further, the alerting device 200 has indentations or ribs 209 around its outer surfaces 210 as illustrated in FIG. 2B in order to minimize the sideways movement of the sensor strip 100 when it is wrapped over the alerting device 200, which will be discuss in details below.

In accordance with the present invention, FIG. 3 illustrates a plan view of the disposable wetness sensor strip 100 when it is in use with an ordinary diaper 300 that needs no further modification. The sensor strip 100 is adhered to the center of a diaper padding 301 by the double-sided adhesive 102 in the peel-and-stick backing of the sensor strip 100. When a sensor strip 100 in matching length and size with the diaper 300 is used, the electrodes 105 in the sensor strip 100 will cover a detection area from the front portion to the rear portion of the diaper 300, corresponding to the region from the groin to the hip of a human body, respectively. In the principle embodiment, a certain length of the sensor strip 100 is protruded out of the diaper's front waistband 302 and is intended to be sufficiently long enough for wrapping the alerting device 200 with a minimum of a turn and half. Further, the sensor strip 100 is designed to function properly regardless of the humidity of the diaper 300 and it does not affect the absorbency of the diaper padding 301.

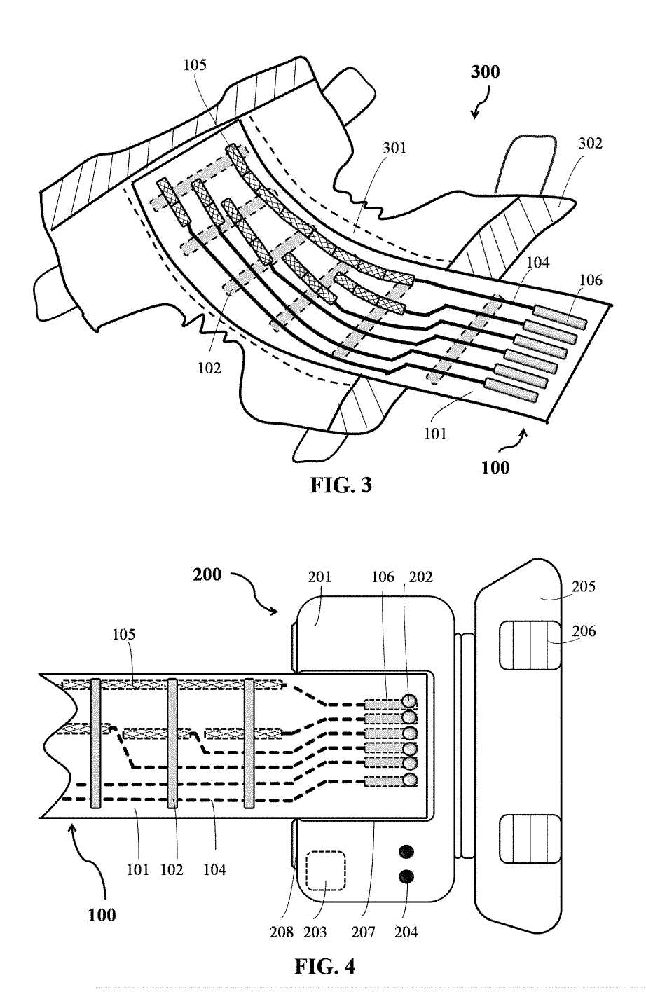

In FIG. 4, it depicts a method of electronically connecting the sensor strip 100 to the alerting device 200 in the principle embodiment. The sensor strip 100 is placed in the indented area 207 of the alerting device 200, wherein the contact terminals 106 and 202, respectively on the sensor strip 100 and the alerting device 200, are properly aligned and connected with each other to form an electrical connection. The electrical connection is then secured by closing the e 9 snap lid 205 and locking the tabs 206 into the locking groove 208 such that the sensor strip 100 is held firmly in place inside the alerting device 200 to prevent any disconnection between the contact terminals 106 and 202 during use. In accordance with the present invention, this electrical connection is temporary such that the alerting device 200 can be released from the sensor strip 100 when a change of diaper is needed and the alerting device 200 is then ready for immediate use.

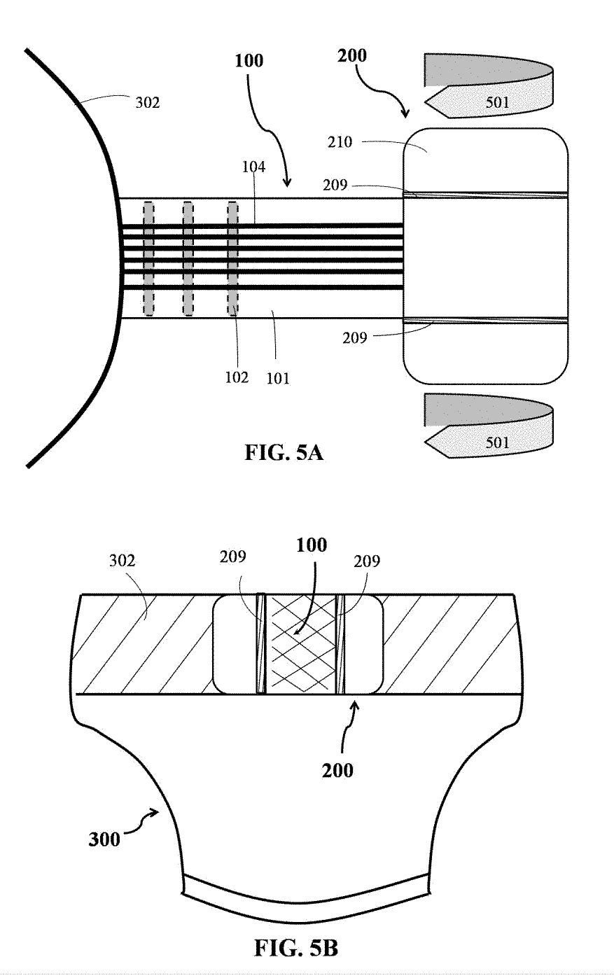

Now referring to FIG. 5A, after connecting the sensor strip 100 to the alerting device 200, it is essential to fasten the alerting device 200 to a place in which the comfort of wearing a diaper with the present invention is not affected. In accordance with the present invention, the extra length of the sensor strip 100 that protrudes out of the diaper's front waistband 302 is wrapped around the alerting device 200 in a circular motion, rotating clockwise 501 (i.e. away from the double-sided adhesive 102 located on the back side of the sensor strip 100) towards the center of the diaper's front waistband 302. The sensor strip 100 that being tightly wrapped around the alerting device 200 is restrained from any sideways movement as its position is bounded by the ribs or indentations 209 that are located around the outer surface 210 of the alerting device 200. In the last rotation turn, the final position of the alerting device 200 is fixed to the outer surface of the diaper's front waistband 302 by the double-sided adhesive 102 such that the alerting device 200 is fastened to the center of the diaper's front waistband 302, as illustrated in FIG. 5B.

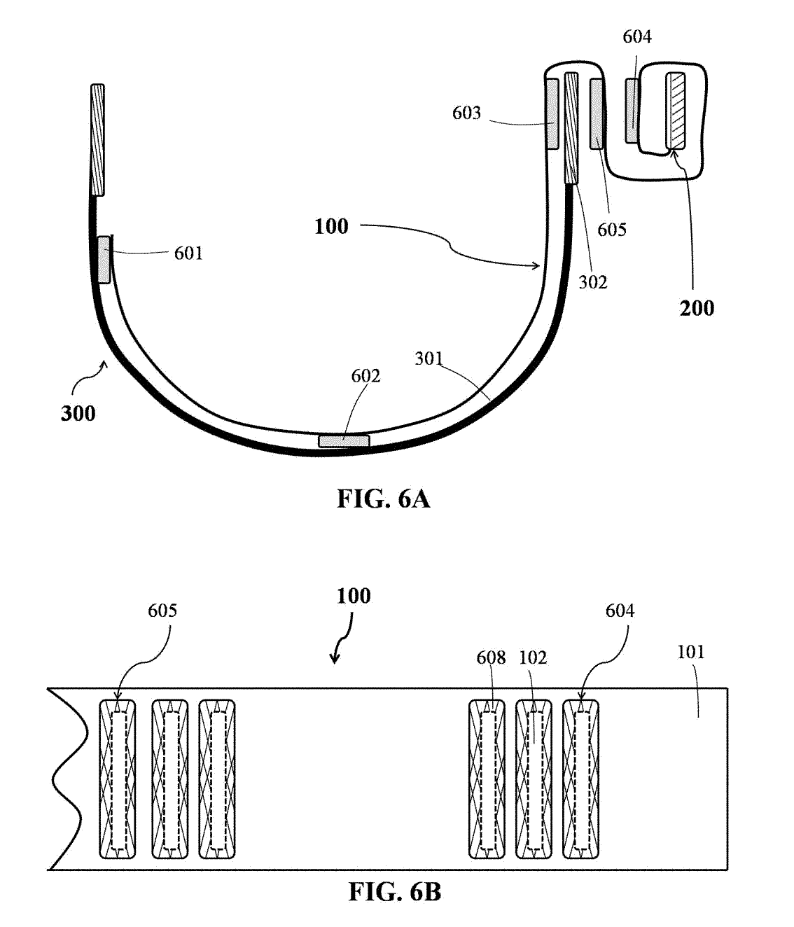

In accordance with the principle embodiment, one of the possible arrangements of the double-sided adhesives 102 is shown in FIG. 6A, wherein the adhesives are located only on the back side of the sensor strip 100. The double-sided adhesives 102 at locations 601 to 603 affix the sensor strip 100 to the diaper padding 301 from a rear area to a front area of the diaper 300. After connecting the sensor strip 100 to the alerting device 200, the sensor strip 100 is tightly wrapped around the alerting device 200 with a minimum of a turn and half, in a clockwise direction 501 towards the diaper's front waistband 302. It is then affixed to the protruded portion of the sensor strip 100 that is in proximity to the diaper's front waistband 302 by the adhesive on location 604. The final position of the alerting device 200 is fixed by the adhesive on location 605, wherein the alerting device 200 that wrapped with the sensor strip 100 is fastened to the outer surface of the diaper's front waistband 302. The ribs or indentation 209 located around the outer surface 210 of the alerting device 200 assist in securing the position of the sensor strip 100 by preventing the sensor strip 100 from slipping to the sides of the alerting device 200. By adopting this wrapping method, wherein the alerting device 200 is wrapped with the sensor strip 100 in a clockwise direction 501, the double-sided adhesives 102 are only needed on one side (i.e. the back side) of the sensor strip 100, thereby the alerting device 200 remains zero contact with any adhesives materials and the alerting device 200 is ready for immediate use when a change of diaper is necessary. In a further embodiment, as illustrated in FIG. 6B, additional double-sided adhesives 102 with individual protective film 608 may be incorporated between locations 604 and 605 to provide a better control of the final position of the alerting device 200.

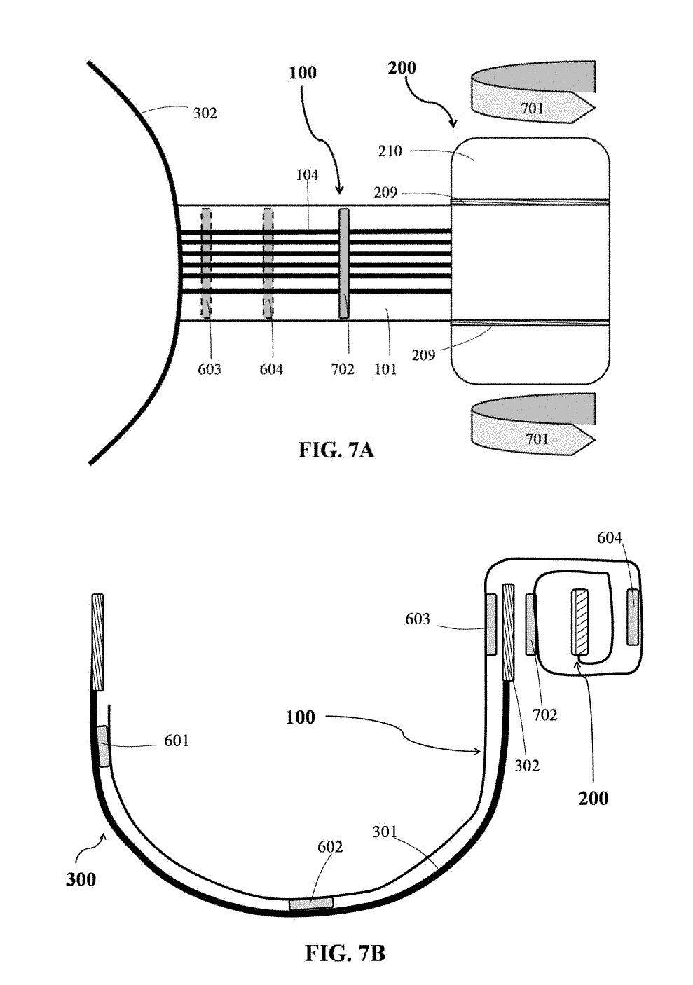

In another embodiment as shown in FIG. 7A, the sensor strip 100 may wrap around the alerting device 200 with a minimum of a turn and half, in an anticlockwise direction 701 towards the diaper's front waistband 302 (i.e. rotating towards the back side of the sensor strip 100). FIG. 7B depicts one of the possible arrangement of the double-sided adhesive 102 when the rotating direction is anticlockwise 701. In addition to the double-sided adhesives 102 distributed on the back side of the sensor strip 100 (e.g. adhesives on locations 601 to 604), at least one adhesive (e.g. adhesive on location 702) is located on the front side of the sensor strip 100 (i.e. on which the side with the electrical circuit printed) in order to fasten the alerting device 200 to the outer surface of the diaper's front waistband 302. By adopting this wrapping method, wherein the alerting device 200 is wrapped with the sensor strip 100 in an anticlockwise direction 701, the double-sided adhesives 102 are needed on both sides of the sensor strip 100 to fasten the alerting device 200 to the outer surface of the diaper 300 while remaining zero contact with any adhesives materials, thereby the alerting device 200 is ready for immediate use when a change of diaper is necessary.

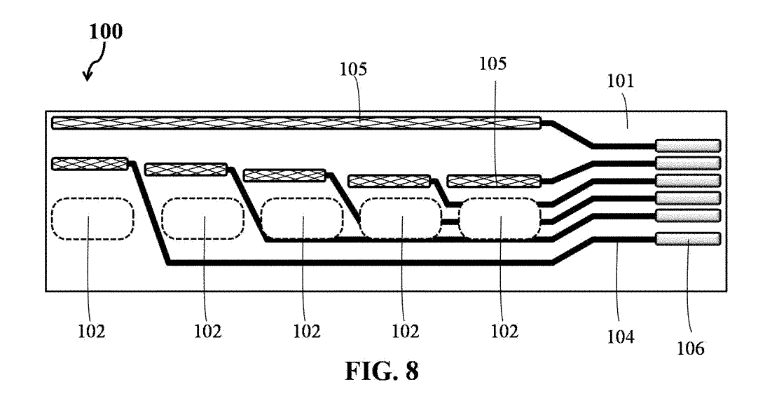

As shown in FIG. 8, in yet another embodiment, the double-sided adhesives 102 may be positioned along the length of the sensor strip 100. In all previous embodiments, the double-sided adhesives 102 do not affect the normal functioning of the diaper 300 and the sensor strip 100, e.g. the flow of urine in the diaper 300 or the conductivity of the electrical circuit on the sensor strip 100. Although the double-sided adhesives 102 are presented as discrete portions on the sensor strip 100 in all of the above embodiments, the double-sided adhesives 102 may equally be presented as continuous strips that run along or across the sensor strip 100. It is also noted that the double-sided adhesives 102 may be arranged in a variety of well known configurations.

Since the sensor strip 100 is designed to have the double-sided adhesive 102 on certain locations in its peel-and-stick backing wherein the locations are adapted to different wrapping directions (i.e. clockwise 501 or anticlockwise 701 direction), the alerting device 200 is not adhesively bonded to the sensor strip 100 since none of the double-sided adhesive 102 touches the alerting device 200. Therefore, when there is a need to change the diaper, the alerting device 200 can be detached from the sensor strip 100 by simply pulling it off from the diaper's front waistband 302 so as to break the double-sided adhesive 102 in-between. The previously wrapped sensor strip 100 is then unrolled and the front section with the contact terminals 106 that is held inside the alerting device 200 is released by unlocking the tabs 206 and opening the snap lid 205 of the alerting device 200. In this particular example, the alerting device 200 can be immediately reused without further procedure such as removing adhesive residue.

In accordance with the present invention, an electrical circuit with a power supply is formed when the sensor strip 100 and the alerting device 200 are electronically connected. When the diaper 300 is clean (i.e. not wetted), the electrical circuit is open and there is no current passing through it. On the contrary, when the diaper 300 is wetted by urine or by other body fluid, which contains not only water but also dissolved salts and ions that acts as electrolytes, one or more of the spaces between the electrodes 105 will be bridged to form a closed electrical circuit that allows current to pass through. Subsequently, the current flow will trigger the alerting device 200 to send out a signal that a diaper change is required. Since the electrodes 105 provide detection areas at various locations along the longitudinal direction of the diaper 300, the wetness locations can then be identified based on the locations of current flow in the urine bridged circuit. In a further embodiment, other electrical circuitry may be implemented to measure the capacitance or voltage change of the wetted diaper such that the degree of wetness of the diaper can be evaluated. Therefore, the present invention allows identification of the wetness location along the diaper as well as the degree of wetness at each of the detection areas.

The invention claimed is:

1. A wetness sensing and alerting system to be attached to a diaper, comprising:
a sensor strip with an adhesive material on at least one side of the said sensor strip; and
an alerting device electrically coupled to the said sensor strip and be wrapped around by the said sensor strip in a way that the adhesive materials on the said sensor strip has no contact with the said alerting device when the said alerting device is fastened to an outer surface of the said diaper.

2. The wetness sensing and alerting system as in claim 1, wherein the diaper is an ordinary diaper that needs no further alterations.

3. The wetness sensing and alerting system as in claim 1, wherein the sensors strip further comprises:
a portion that is long enough to wrap around the alerting device with a minimum of a turn and half after the said sensor strip is adhesively bonded to a diaper padding from a rear area to a front area of the said diaper.

4. The wetness sensing and alerting system as in claim 1 wherein:
the adhesive material is located only on one side of the sensor strip when the said alerting device is wrapped around by the said sensor strip in a circular motion that rotates in a clockwise direction towards the waistband of the said diaper.

5. The wetness sensing and alerting system as in claim 1 wherein:
the adhesive material is located on both sides of the sensor strip when the said alerting device is wrapped around by the said sensor strip in a circular motion that rotates in a anticlockwise direction towards the waistband of the said diaper.

6. The wetness sensing and alerting system as in claim 1, wherein the alerting device further comprising:
an outer surface thereof contoured to provide a plurality of ribs or an indentation to minimize sideways movement of the said sensor strip once the said sensor strip is electrically coupled to the said alerting device and wrapped over the said alerting device.

7. The wetness sensing and alerting system as in claim 1, wherein the alerting device further comprising:
a lid with locking means to secure the sensor strip in place when the said sensor strip is electrically coupled to the said alerting device.

8. The wetness sensing and alerting system as in claim 1, wherein the alerting device further comprises:
an electrical connection means for electrically coupled to an electrical connection means on the sensor strip;
an electronic means to receive and process signals from the said connected sensor strip and to provide an alarm signal by a wireless means; and
a power storage means.

9. The wetness sensing and alerting system as in claim 1, wherein the sensor strip further comprises:
an electric circuit with a plurality of conductive band each having a contact terminal in one end and an electrode as a wetness detector in the other end, such that the said sensor strip covers an detection area from a rear area to a front area of the said diaper, whereby, in use, the said electric circuit is closed when the said plurality of electrodes are bridged by urine or other body fluid to produce an alarm signal for diaper-change.

10. The wetness sensing and alerting system as in claim 9, wherein the electrodes are responsive to urine or other body fluid.

11. The wetness sensing and alerting system as in claim 9, wherein the electric circuit further comprises an electrical circuitry means to measure a capacitance or voltage change of the diaper to identify a wetness location and a degree of wetness at each of the said wetness location.

12. A wetness sensing and alerting system as in claim 1, wherein the alerting device is fastened to the outer surface of a front waistband of the diaper.

13. The wetness sensing and alerting system as in claim 1, wherein the alerting device further comprises an indented area with a width and an indented depth that match with the sensor strip such that the sensor strip is fitted into the said indented area.

14. The wetness sensing and alerting system as in claim 1, wherein the alerting device is a disposable or a non-disposable component in the system.

15. The wetness sensing and alerting system as in claim 1, wherein the sensor strip is temporarily electrically coupled to the alerting device such that the said alerting device is released from the said sensor strip ready for immediately use.

16. The wetness sensing and alerting system as in claim 1 wherein the adhesive material on the sensor strip is double-sided adhesives that are covered by a continuous piece or individual piece of protective film(s) prior to use.

17. The wetness sensing and alerting system as in claim 1 wherein the adhesive material on the sensor strip is in a form of discrete portions or continuous strips that run along or across the sensor strip.
